(19) United States
(12) Patent Application Publication
(10) Pub. No.: US 2016/0281126 A1
(43) Pub. Date: Sep. 29, 2016

(54) High-Throughput Yeast-Aging Analysis (HYAA) Chip For Performing Yeast Aging Assays

(71) Applicant: Lidong Qin, Houston, TX (US)

(72) Inventors: Lidong Qin, Houston, TX (US); Myeong Chan Jo, Houston, TX (US); Weiwei Dang, Houston, TX (US)

(73) Assignees: Lidong Qin, Houston, TX (US); Myeong Chan Jo, Houston, TX (US); Baylor College of Medicine, Houston, TX (US)

(21) Appl. No.: 15/173,713

(22) Filed: Jun. 5, 2016

(51) Int. Cl. C12Q 1/24; C12M 3/06; C12M 1/34; B01L 3/00

(52) U.S. Cl. CPC C12Q 1/24; B01L 3/502715; B01L 3/502761; C12M 23/16; C12M 41/46; B01L 2300/0877; B01L 2300/123; B01L 2300/0867; B01L 2200/0647; B01L 2300/0816; B01L 2300/0864; B01L 2300/0819; B01L 2300/0654; G01N 2333/39; G01N 2800/7042

(57) ABSTRACT

An improved technique for studying the molecular mechanisms of aging in eukaryotic cells utilizes an efficient, high-throughput microfluidic single-cell analysis chip in combination with high-resolution time-lapse microscopy. A High-throughput Yeast Aging and Analysis (HYAA) Chip has a plurality of discrete microfluidic channels grouped into a number of modules. Each module has a single medium inlet and a single medium outlet. Each channel in a module has a microfluidic chamber having a plurality of single-cell trapping structures, and features a sample inlet for introducing cells into the flow of medium through the chamber. This innovative design enables the determination of the yeast replicative lifespan in a high throughput manner.

### TECHNICAL FIELD

The present invention, in some embodiments thereof, relates broadly to methods and apparatus for capturing (trapping) and cultivating individual mother cells, while shedding resulting daughter cells (buds), using microfluidic technology, and observing growth of the mother cells in different mediums and under different conditions.

### BACKGROUND

Aging and age-associated diseases are becoming one of the fastest growing areas of epidemiology in most developed countries. Identification of molecular mechanisms that lead to the development of interventions to delay the onset of age-associated diseases could have tremendous global impacts on public health. The budding yeast, Saccharomyces cerevisiae (S. cerevisiae, used for winemaking, baking, and brewing since ancient times) was the first eukaryotic genome to be sequenced, and has been instrumental in discovering molecular pathways involved in all aspects of eukaryotic cells. S. cerevisiae is an important model for discovering evolutionarily conserved enzymes that regulate aging, such as Sir2 and Tor1. S. cerevisiae cells are typically round to ovoid, 5-10 μm in diameter, and reproduce by a division process known as budding.

Understanding the molecular mechanisms that regulate aging and age-associated diseases is an instrumental step toward designing interventions that delay the onset of diseases and physiological changes linked to aging, which is a leading risk factor for many diseases. Considerable research effort has focused on uncovering the molecular mechanisms of aging and their contributions to age-associated diseases. The replicative lifespan measurement of yeast cells has become a general method for mechanistic studies of aging processes, and has been used to identify genes and pathways associated with longevity that are conserved among all eukaryotes. Saccharomyces cerevisiae has been an important model for studying the molecular mechanisms of aging in eukaryotic cells. However, the laborious and low-throughput methods of current yeast replicative lifespan assays limit their usefulness as a broad genetic screening platform for research on aging.

Current yeast aging research is fraught with technical challenges including labor-intensive and time-consuming experimentation, low-throughput data collection, discontinuous tracking, and the lack of reliable single-cell assays. For example, yeast replicative lifespan (RLS) is typically determined by manually separating the daughter cells from a mother cell on a petri dish with a microscope-mounted glass needle, and counting the number of divisions throughout the life of the cell. Tens or hundreds of cells per strain have to be dissected and counted to determine whether the lifespans of two strains are statistically different. This method has not changed appreciably since the initial discovery of yeast replicative aging in 1959. A well-trained yeast dissector can monitor and handle no more than 300 cells at once, and a typical lifespan experiment usually thus lasts ˜4 weeks. Most lifespan experiments include an overnight 4° C. incubation everyday throughout the experiment for practical purposes, adding another factor that can complicate data interpretation. This tedious and low-throughput procedure has substantially hindered progress. Therefore, new strategies are required to take advantage of the power of yeast genetics and apply high-throughput unbiased genetic screen approaches to yeast aging research.

Microfluidics is an emerging multidisciplinary field intersecting engineering, physics, chemistry, biochemistry, nanotechnology, and biotechnology, with practical applications to the design of systems in which low volumes of fluids are processed to achieve multiplexing, automation, and high-throughput screening. Advances in microfluidics technology are revolutionizing molecular biology procedures for enzymatic analysis (e.g., glucose and lactate assays), DNA analysis (e.g., polymerase chain reaction and high-throughput sequencing), and proteomics. The basic idea of microfluidic biochips is to integrate assay operations such as detection, as well as sample pre-treatment and sample preparation on one chip.

### SUMMARY

It is an object of the invention to provide an improved technique (method and apparatus) for studying the molecular mechanisms of aging in eukaryotic cells.

According to the invention, generally, the improved technique utilizes an efficient, high-throughput microfluidic single-cell analysis chip in combination with high-resolution time-lapse microscopy. This innovative design enables, to our knowledge for the first time, the determination of the yeast replicative lifespan in a high throughput manner. Morphological and phenotypical changes during aging can also be monitored automatically with a much higher throughput than previous microfluidic designs. The techniques disclosed herein allow for highly efficient trapping and retention of mother cells, determination of the replicative lifespan, and tracking of yeast cells throughout their entire lifespan. Using the high-resolution and large-scale data generated from the high-throughput yeast aging analysis (HYAA) chips, particular longevity-related changes in cell morphology and characteristics are readily investigated, including critical cell size, terminal morphology, and protein sub-cellular localization. In addition, because of the significantly improved retention rate of yeast mother cell, the HYAA Chip was capable of demonstrating replicative lifespan extension by calorie restriction.

According to some embodiments (examples) of the invention, a module for isolating and culturing a plurality of single cells may comprise: a thin sheet of flexible or semi-rigid material; a medium inlet, a medium outlet, and a channel extending in fluid communication between the medium inlet and the medium outlet; a chamber disposed the channel; a plurality of single-cell trapping structures disposed in the chamber; and a sample inlet for introducing cells into a flow of medium through the chamber. The thin sheet of flexible or semi-rigid material may be selected from the group consisting of polydimethylsiloxane (PDMS), PMMA (poly(methyl methacrylate)), PS (polystyrene), and PC (polycarbonate). The thin sheet of flexible or semi-rigid material may have a thickness of approximately 8 μm. The thin sheet of flexible or semi-rigid material may be molded to have channels and single-cell trapping structures on a front surface thereof. The dimensions of the trapping structures may be optimized to ensure that at least one of the following conditions are met: (i) only a single cell is captured in each trapping structure; (ii) the trapped cells are stably retained in the trapping structure during the entire course of an aging experiment; and (iii) the trapping structure does not pose a spatial constraint to cell size increase during aging.

A high-throughput yeast-aging analysis (HYAA) chip may comprise a plurality of modules for isolating and culturing a plurality of single cells. Each module may have a single medium inlet disposed on one side thereof, and a single medium outlet disposed on an opposite side thereof. The modules may be disposed one above the other on a surface of the thin sheet of flexible or semi-rigid material so that their inlets are all oriented to the one side of the sheet and their outlets are oriented to an opposite side of the sheet. Each module may include a plurality of channels branched from its single medium inlet and merged into its single medium outlet. The channels may be parallel with one another. A plurality of single-cell trapping structures may be disposed in each of the chambers, so as to be capture single cells from the flow of medium through the chamber. The trapping structures may be arranged in an array having a number of columns of trapping structures disposed vertically, one above the other, with spaces therebetween. The trapping structures of one column may be offset vertically from the trapping structures of an adjacent column to facilitate flow of medium and cells through the array. The trapping structures may be cup-shaped, having an inlet and an outlet. The inlets of the trapping structures may be larger than the outlets of the trapping structures. A width of the inlets of the trapping structures may be approximately 6 μm. A width of the outlets of the trapping structures may be approximately 3 μm. 14. A height of the trapping structures may be approximately 5 μm. A plurality of trapping structures may be arranged in an array of columns and rows. A column spacing may be equal to or smaller than a row spacing to ensure high trapping efficiency and minimal channel obstruction by daughter cells removed from the trapped mother cells.

According to some embodiments (examples) of the invention, a method of isolating and culturing a plurality of single cells may comprise: providing a module comprising: a medium inlet, a medium outlet, and a channel extending in fluid communication between the medium inlet and the medium outlet; a chamber disposed the channel; a plurality of single-cell trapping structures disposed in the chamber; and a sample inlet for introducing cells into a flow of medium through the chamber; and may further comprise introducing a liquid medium continuously through the medium inlet; injecting suspended yeast cells through the sample inlet; and trapping individual mother cells in the single-cell trapping structures. The trapped mother cells may be cultivated with continuous medium flow. As the trapped cells mother cells grow and bud, and daughter cells may be produced and may be detached from their mother cells, removing the daughter cells by the medium flow. The development of the mother cells may be tracked over their entire lifespan in a single experiment using high-resolution multi-position time-lapse microscopy.

### Significance (Advantages)

Advancing our understanding of the underlying molecular mechanisms of aging, as well as their contributions to age associated diseases, may have a profound impact on public health. Studying the replicative aging phenomenon in the budding yeast Saccharomyces cerevisiae has led to significant findings on how aging is regulated by evolutionarily-conserved enzymes and molecular pathways. The microfluidic system disclosed herein enables the visualization and analysis of the complete replicative lifespan of a large number of single yeast cells.

This system overcomes current technical challenges in low throughput yeast lifespan analysis by providing a fast, high throughput, and accurate analytical method at the single-cell level. This approach opens a new avenue for aging and longevity research using yeast genetic screens.

Using the HYAA Chip disclosed herein allows immobilization of single yeast cells and removal of newly-budded daughter cells without losing trapped mother cells, in a highly efficient manner. The microfluidic platform combines the HYAA Chip with high-resolution multi-positioning time-lapse microscopy. This approach offers an unparalleled method for aging studies. First, the platform allows fully automated tracking of the entire lifespan for several thousand individual cells in a single experiment with high spatiotemporal resolution. This platform saves labor and time. It should be noted that the device cannot selectively trap virgin cells at the beginning of the experiment. However, in a typical log-phase culture, ˜80% of total cells are virgin and 12% have only budded once. Thus, the lack of virgin cell selection should not have a significant effect on the final lifespan results. Second, the HYAA Chip can be easily multiplexed by connecting multiple channels. This platform enables simultaneous analysis of multiple strains and multiple media, resulting in high-throughput quantification of longevity. Third, fluorescent imaging of single cells during the entire aging process offers high spatiotemporal resolution and high-throughput examination of the aging phenotype, including organelle morphology, gene expression, and protein localization. Therefore, genetic or environmental factors that regulate lifespan can be investigated at the single cell level. Finally, this platform allows cells to be maintained under a constant growth condition in the microfluidic channel throughout their entire lifespan, thereby minimizing variations introduced by operators and the environment. These capabilities effectively remove the barriers of existing lifespan assays that have hindered high throughput aging studies in yeast.

Other features and aspects of the invention will become apparent from the following detailed description, taken in conjunction with the accompanying drawings, which illustrate, by way of example, the features in accordance with embodiments of the invention. The summary is not intended to limit the scope of the invention, which is defined solely by the claims attached hereto.

### BRIEF DESCRIPTION OF THE DRAWINGS

The present invention, in accordance with one or more various embodiments, is described in detail with reference to the following figures. The drawings are provided for purposes of illustration only and merely depict typical or example embodiments of the invention. These drawings are provided to facilitate the reader's understanding of the invention and shall not be considered limiting of the breadth, scope, or applicability of the invention. It should be noted that for clarity and ease of illustration these drawings are not necessarily made to scale.

Some of the figures included herein illustrate various embodiments of the invention from different viewing angles. Although the accompanying descriptive text may refer to such views as “top,” “bottom” or “side” views, such references are merely descriptive and do not imply or require that the invention be implemented or used in a particular spatial orientation unless explicitly stated otherwise.

FIG. 1A is a diagram (top view) of an overall High-throughput Yeast-Aging Analysis (HYAA) Chip which may be used for performing yeast aging assays.

FIG. 1B is a diagram (top view) of a module of the HYAA chip.

FIG. 1C is a diagram (top view) of a portion of a microfluidic chamber in a module having a plurality of single-cell trapping structures (“traps”)

FIG. 1D is a diagram (perspective view) of a single trap.

FIG. 2A is a diagram (top view) of a single cell trapped in a trap.

FIG. 2B is a diagram (top view) of the single cell culturing & budding.

FIG. 2C is a diagram (top view) of the bud washing away from the trap.

The figures are not intended to be exhaustive or to limit the invention to the precise form disclosed. It should be understood that the invention can be practiced with modification and alteration, and that the invention be limited only by the claims and the equivalents thereof.

### DESCRIPTION

From time-to-time, the present invention is described herein in terms of example environments. Description in terms of these environments is provided to allow the various features and embodiments of the invention to be portrayed in the context of an exemplary application. After reading this description, it will become apparent to one of ordinary skill in the art how the invention can be implemented in different and alternative environments.

Unless defined otherwise, all technical and scientific terms used herein have the same meaning as is commonly understood by one of ordinary skill in the art to which this invention belongs.

All patents, applications, published applications and other publications referred to herein are incorporated by reference in their entirety. If a definition set forth in this section is contrary to or otherwise inconsistent with a definition set forth in applications, published applications and other publications that are herein incorporated by reference, the definition set forth in this document prevails over the definition that is incorporated herein by reference.

Any dimensions set forth herein should be regarded as exemplary and approximate, unless otherwise indicated in the description, and may be interpreted to indicate the relative scale of various elements which may be described.

Microfluidic system designs for studying yeast aging have been reported previously; however, they did not permit assays of yeast replicative lifespan because they are unable to track the entire lifespan of mother cells due to the low efficiency in removing daughter cells. The designs reported previously can track trapped mother cells for only up to 18 hours and for up to eight divisions. Although another system could track the whole lifespan of trapped mother cells, the retention rate of these cells in the original traps over the time course was ˜40-60%. More than 40% of monitored cells were lost before reaching the end of their lifespan. In addition, the systems were not designed to analyze multiple strains simultaneously.

In contrast to these previous designs, the HYAA Chip disclosed herein features completely redesigned trapping structures with a greater than 90% retention rate, as well as the capability to assay multiple strains (in different mediums) in the same experiment, resulting in high-throughput quantification of the replicative lifespan in yeast. To our knowledge, these features make our system the first truly high-throughput and reliable platform for examining the replicative lifespan, which sets us apart from any of the previously reported designs.

According to the invention, generally, a High-throughput Yeast Aging and Analysis (HYAA) Chip has a plurality of discrete microfluidic channels (“channels”) grouped into a number of modules. Each module has a single medium inlet and a single medium outlet. Each channel in a module has a microfluidic chamber having a plurality of single-cell trapping structures (“traps”), and features a sample inlet for introducing cells into the flow of medium through the chamber.

In a typical aging experiment, a fresh medium may be introduced continuously through the medium inlet, and suspended yeast cells may be injected through the sample inlet connected directly to each channel. As a result, each channel may be occupied by one strain, allowing the simultaneous analysis of multiple strains in the same experiment. This design allows aging analysis of a plurality (such as up to 16 strains) in a single type of medium, or four strains in up to four different media.

### Performing the Procedure

Generally, a medium flows through the channels, and through and around the traps. Suspended cells may be introduced into the flow, and individual ones of the cells become trapped in individual ones of the traps. Trapped cells (“mother cells”) develop, and buds (“daughter cells”) are shed and carried away by the flowing medium. Various experiments and procedures may then be performed on the mother cells.

In a typical aging experiment, fresh medium is introduced continuously through the medium inlet, and suspended yeast cells are injected through the sample inlet connected directly to each channel. As a result, each channel is occupied by one strain, allowing the simultaneous analysis of multiple strains in the same experiment. This design allows aging analysis of up to 16 strains in a single type of medium or four strains in up to four different media. There may be a total of 8,320 single-cell traps in each device (HYAA Chip). It should be noted that the number of single cells to be tracked for testing can be adjusted by tuning the microscope program and experimental conditions, such as time-lapse interval, the number of capture positions, and the type of objective lens.

Once cell trapping is complete, the cultivation of trapped cells may be conducted with continuous medium flow, such as with YTD (Yeast extract-Peptone-Dextrose) media at 30° C. As the trapped cells grow and bud (are cultured), daughter cells are produced, detached from their mother cells, and then removed by the medium flow. Independent of the position from which the daughter cells budded from the mother cell surface, the daughter cells are pushed by the trap structure shape into two positions within the trap: the main larger opening against the flow direction or the smaller outlet opening. The daughter cells are washed away continuously from the mother cells by the medium, which flows at a rate that is significantly lower than that used for yeast cell-loading. The continuous medium flow removes daughter cells throughout the entire lifespan of the mother cells. By combining the HYAA-Chip with high-resolution multi-position time-lapse microscopy, the rapid and automated tracking of up to thousands of single cells may be accomplished over their entire lifespan in a single experiment.

### Automated Tracking of the Whole Lifespan of Single Yeast Cells

To track the entire lifespan of budding yeast cells, the HYAA-Chip was mounted onto the stage of an inverted microscope equipped with an incubator system

The HYAA Chip enables a large-scale replicative lifespan assay of several (such as up to 16) strains in a single experiment. The techniques disclosed herein can be used to complete automated whole-lifespan tracking of a large number of single cells in 3-4 days instead of 3-4 weeks, as may typically be required for the conventional microdissection method, thereby greatly reducing the labor and time required for each experiment. Automated tracking of aging cells in the HYAA Chip allow for the tracking of cell-cycle dynamics in single cells

Various experiments may be performed and assayed, such as on BY4742 yeast cells. Some experiments have demonstrated that the HYAA Chip lifespan assay accurately determined that three mutants may have a shortened lifespan (bre1Δ, chl1Δ, and rpn4Δ) and nine mutants may have a longer lifespan (fob1Δ, hsp104Δ, idh1Δ, rp122aΔ, sas2Δ, sip2Δ, tma19Δ, tor1Δ, and ubr2Δ) than the WT strain. These results not only provide convincing evidence for the high efficiency and high-throughput capability of this novel yeast replicative lifespan assay, but also demonstrate that lifespan measurements made by the HYAA Chip were consistent with those obtained using the conventional microdissection approach.

The effects of calorie restriction may be studied. Calorie restriction (CR), which involves a dietary regimen low in calories without malnutrition, extends the lifespan of most model organisms including yeast, worms, flies, and mammals. CR is commonly performed in yeast by reducing the glucose concentration in otherwise glucose rich medium. To examine if the longevity effect of CR could be detected in this high-throughput microfluidic setting, lifespan assays were performed with the HYAA Chip using the synthetic complete (SC) media containing 2.0% (normal condition), 0.5% (moderate CR), and 0.05% (severe CR) (wt/vol) glucose. Results indicated that the WT lifespan was progressively and significantly extended as glucose concentration was reduced. The effect of CR on the average cell-cycle time was small and insignificant, indicating that cells were not starved under these conditions. However, significant variation in cell-cycle time was observed for cells under CR conditions, likely due to oscillations of cellular metabolic states under CR.

### Benefits

The High-throughput Yeast-Aging Analysis (HYAA) Chip (or HYAA-Chip) disclosed herein, including its design and strategy of use, is innovative comparing to traditional yeast aging assays and recent microfluidics designs, and may provide the following benefits and features.

(a) The HYAA-Chip allows not only capturing up to great number (such as 8,000) of individual yeast mother cells, with 96% capture efficiency, but also features automated-releasing of daughter cells (or buds) continuously, until all of the captured mother cells die.

(b) The HYAA-Chip includes arrays of capture structures. The design of the capture structure (cup-shaped with the height, main opening, and outlet opening of 5, 6, and 3 μm, respectively, is innovative to ensure: (i) automated-releasing of daughter cells by the outlet opening, (ii) only as single mother cell capture in each structure, and (iii) prevention of a spatial constraint to cell size increase during mother cell aging and growth.

(c) The HYAA-Chip provides the stable immobilization of the captured mother cells with quite high retention rate of up to 92% for quite long-term experiments of up to 96 hours (h).

(d) The HYAA-Chip allows mother cells to be maintained under constant growth conditions throughout the entire lifespan of mother cells by supplying a continuous flow of fresh medium.

### An Implementation of the HYAA Chip

An exemplary design and working mechanism of an HYAA Chip for studying aging in yeast will now be described, with respect to the following figures.

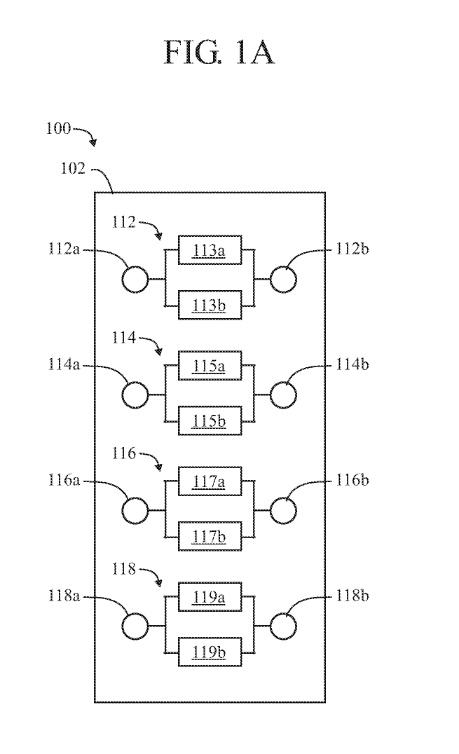

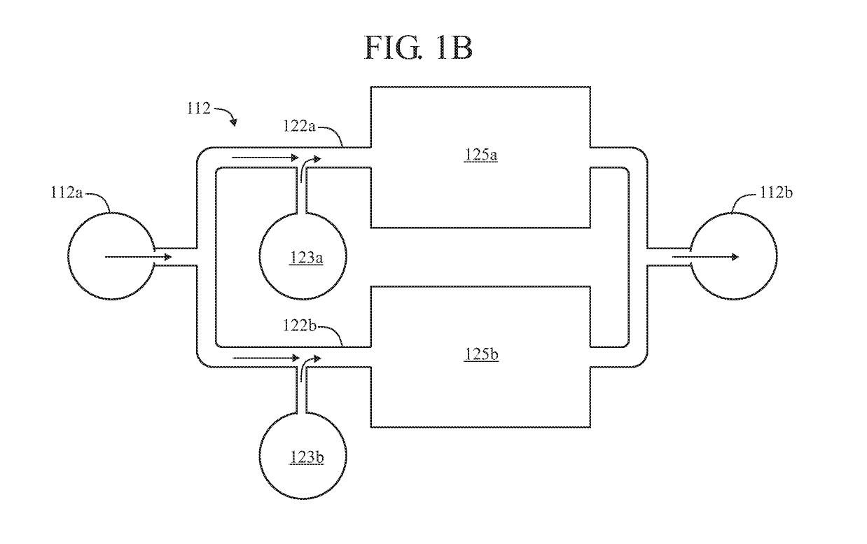

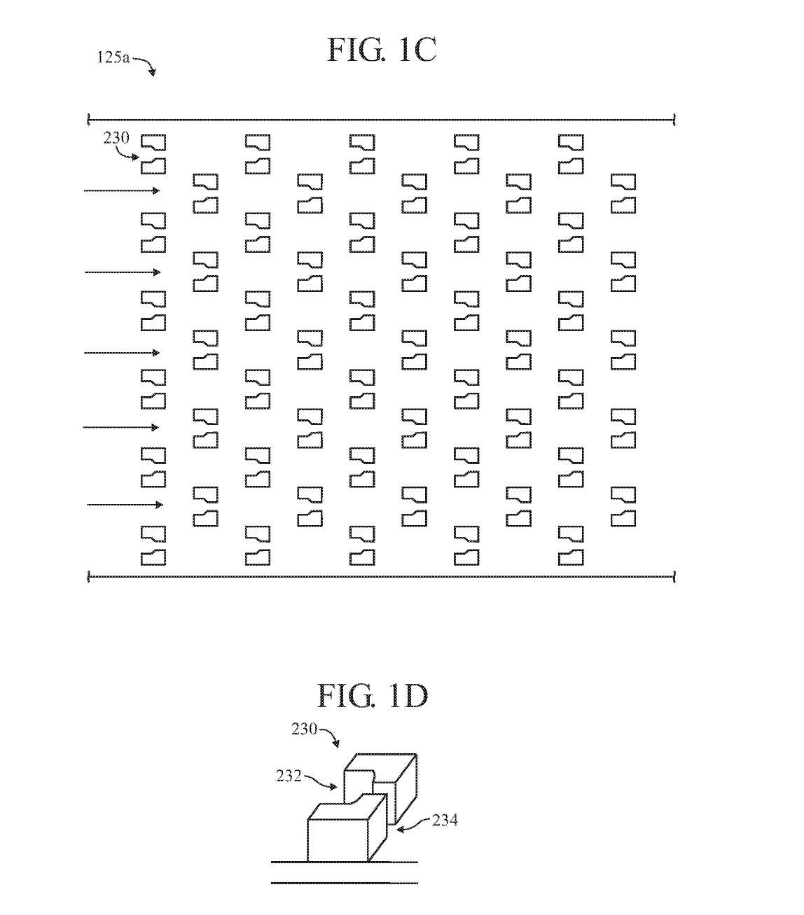

FIG. 1A illustrates a High-throughput Yeast-Aging Analysis (HYAA) Chip 100. The chip is shown as rectangular, having a height of approximately 40 mm and a width of approximately 20 mm.

The HYAA chip (or simply “chip”) may comprise a thin sheet 102 of flexible or semi-rigid material selected from the group consisting of polydimethylsiloxane (PDMS), PMMA (poly(methyl methacrylate)), PS (polystyrene), and PC (polycarbonate), may have a thickness of approximately 8 μm, and may be molded to have channels and single-cell trapping structures on a front surface thereof, as described in greater detail hereinbelow.

The chip may have four modules 112, 114, 116, 118 disposed on the front surface thereof. Each module may have a single medium inlet disposed on one side (left, as viewed) thereof, and a single medium outlet disposed on an opposite side (right, as viewed) thereof. As illustrated, the module 112 has a medium inlet 112a and a medium outlet 112b, the module 114 has a medium inlet 114a and a medium outlet 114b, the module 116 has a medium inlet 116a and a medium outlet 116b, the module 118 has a medium inlet 118a and a medium outlet 118b. The modules may be disposed one above the other, as illustrated, so that their inlets are all oriented to the left (as viewed) and their outlets are oriented to the right (as viewed).

Each module may include a number (such as two, as shown, or more) of channels branched from (disposed in fluid communication between) its single medium inlet and merged into its single medium outlet. The module 112 is shown with two channels 113a and 113b extending in fluid communication between its inlet 112a and outlet 112b. The module 114 is shown with two channels 115a and 115b extending in fluid communication between its inlet 114a and outlet 114b. The module 116 is shown with two channels 117a and 117b extending in fluid communication between its inlet 116a and outlet 116b. The module 118 is shown with two channels 119a and 119b extending in fluid communication between its inlet 118a and outlet 118b.

FIG. 1B shows, in greater detail, an exemplary one of the modules—the module 112—which may be representative of the other modules 114, 116, 118. The module 112 has a single medium inlet 112a and a single medium outlet 112b, and a number (such as two, but may be more) of microfluidic channels (“channels”) 122a and 122b extending in fluid communication between its inlet 112a and its outlet 112b. The channels 122a and 122b may be parallel with one another.

Generally, a fluid introduced into the inlet 112a will flow through the channels 122a and 122b, and from there into the outlet 112b. The arrows show the direction of flow. The fluid may be a medium containing nutrients for cells being assayed in the module.

A sample inlet 123a is associated with and in fluid communication with the channel 122a, at an upstream portion thereof (e.g., at the inlet end of the channel) and allows for a suspension of cells to be introduced into medium flowing in the channel 122a between the inlet 112a and the outlet 112b. Similarly, a sample inlet 123b is associated with and in fluid communication with the channel 122b, at an upstream portion thereof (e.g., at the inlet end of the channel) and allows for a suspension of cells to be introduced into medium flowing in the channel 122b between the inlet 112a and the outlet 112b.

A downstream portion of the channel 122a may comprise and may be referred to as a microfluidic chamber 125a, and may be much wider than the upstream portion of the channel 122a. Similarly, a downstream portion of the channel 122b may comprise and may be referred to as a microfluidic chamber 125b, and may be much wider than the upstream portion of the channel 122b.

The modules 114, 116 and 118 may similarly have a single medium inlet and a single medium outlet, and a number (such as two, but may be more) of channels extending in fluid communication between their respective inlets and their outlets. Each module 114, 116 and 118 may also have sample inlets and microfluidic chambers in the manner described hereinabove.

FIG. 1C shows, in greater detail, a portion of the microfluidic chamber 125a, as representative of other microfluidic chambers. A plurality (such as 520) of single-cell trapping structures (or “traps”) 230 may be disposed in the microfluidic chamber, so as to be capture single cells from the flow of medium through the chamber. The several traps may be arranged in an array having a number of columns of traps disposed vertically, one above the other, with spaces therebetween, and the traps of one column may be offset vertically from the traps of an adjacent column to facilitate flow of medium and cells through the array. FIG. 1C shows a representative five columns having five traps each, and five columns having five traps each. There may be different numbers of traps in each column, and there may be many more columns. The arrows show the direction of fluid flow through the chamber.

FIG. 1D shows a representative one of the traps 230. The traps may be cup-shaped (or funnel-shaped) having an inlet (main opening) 232 and an outlet 234. The inlet of the trap may be larger than (such as twice as large as) the outlet of the trap. The width of the inlet of the trap may be approximately 6 μm. The width of the outlet of the trap may be approximately 3 μm. The depth (or height) of the trap may be comparable to the width of the inlet, such as approximately 5 μm.

The dimensions of the trap may be empirically optimized to ensure that the following conditions are met: (i) only a single cell is captured in each trap; (ii) the trapped cells are stably retained during the entire course of an aging experiment; and (iii) the trap does not pose a spatial constraint to cell size increase during aging.

Referring back to FIG. 1C, the spacing between traps in the array is an important parameter for efficient single-cell trapping. The column spacing may be equal to or smaller than the row spacing for higher trapping efficiency. For example the column spacing may be 12 μm, and the row spacing may also be 12 μm. This may ensure high single-cell trapping efficiency and minimal channel obstruction by daughter cells removed from the trapped mother cells.

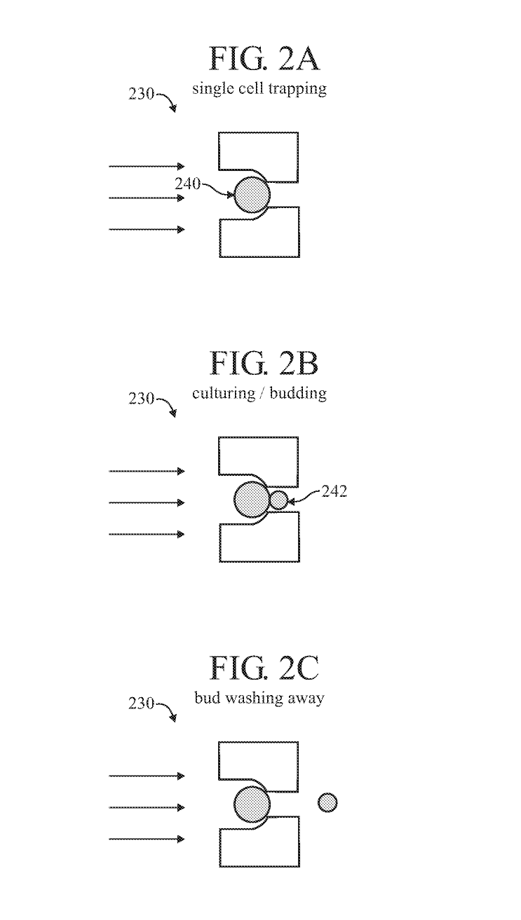

In an example of a single yeast cell showing the working mechanism procedure, FIG. 2A shows a single cell 240 having been trapped in the inlet (main opening) of a trap 230. Medium flow is indicated by the arrows. FIG. 2B shows the single cell (mother cell) 240 having formed a bud (daughter cell) 242 which, in this example, is located in the outlet of the trap. The daughter cell may form in available space in the inlet of the trap, ahead of the mother cell. In either case, the daughter cell 242 will be washed away by the flow of medium, as shown in FIG. 2C.

Some Contrasts with Previous Systems

Microfluidic devices have been developed to capture yeast cells for high-resolution imaging analysis during vegetative growth. Recently, such devices have been designed that enable the tracking of yeast cells throughout their lifespan, making it possible to record and study cellular phenotypic changes during aging. However, many issues prevent the use of microfluidic devices in a high-throughput manner for lifespan screens. First, although the time required to monitor the entire lifespan of the yeast cell has been dramatically reduced, the throughput is limited to 1-4 channels per device. Second, mother cells were immobilized underneath soft elastomer [polydimethylsiloxane (PDMS)] micropads. Although several hundred trapping micropads can be assembled for each microfluidic channel, such a trap design suffers from a low retention rate of ˜30% by the end of the lifespan; this seriously limits the number of usable cells in the lifespan calculation to ˜100, which restricts statistical significance of the lifespan analysis. Third, the ability for trapping micropads to retain old cells depends on the larger size of old cells compared with young cells. However, old cells often generate large daughter cells that also become trapped by the micropads. Fourth, the micropad design often allows more than one cell to be trapped; multiple cells can be trapped underneath one micropad, whereas no cells are trapped under others. Finally, in some previous designs, cell-surface labeling and chemical modification of the device are required, which has proven to be technically challenging for fabrication and to introduce adverse effects on replicative lifespan.

The HYAA Chip disclosed herein may solve all of the above-described challenges and limitations. This innovative design can trap up to 8,000 individual yeast cells in cup-shaped PDMS structures evenly distributed to 16 discrete channels; captured cells are cultivated and aged as fresh medium continuously flows through, which removes newly budded daughter cells. The HYAA Chip provides automated whole-lifespan tracking with fine spatiotemporal resolution and large-scale data quantification of single yeast cell aging by combining simple fabricated microfluidics with high-resolution time-lapse microscopy. The HYAA-Chip is label free, independent of size differences between mother and daughter cells, has up to 96% single-cell trapping efficiency, and up to 92% retention rate for the initially trapped mother cells.

### Appendices

Appended hereto, and forming part of the disclosure hereof, is a document entitled High-throughput analysis of yeast replicative aging using a microfluidic system, by Myeong Chan Jo, Wei Liu, Liang Gua, Weiwei Dang, and Lidong Qin

Although the invention is described above in terms of various exemplary embodiments and implementations, it should be understood that the various features, aspects and functionality described in one or more of the individual embodiments are not limited in their applicability to the particular embodiment with which they are described, but instead can be applied, alone or in various combinations, to one or more of the other embodiments of the invention, whether or not such embodiments are described and whether or not such features are presented as being a part of a described embodiment. Thus the breadth and scope of the present invention should not be limited by any of the above-described exemplary embodiments.

What is claimed is:

1. A module for isolating and culturing a plurality of single cells, comprising:
a thin sheet of flexible or semi-rigid material;
a medium inlet, a medium outlet, and a channel extending in fluid communication between the medium inlet and the medium outlet;
a chamber disposed the channel;
a plurality of single-cell trapping structures disposed in the chamber; and
a sample inlet for introducing cells into a flow of medium through the chamber.

2. The module of claim 1, wherein the thin sheet of flexible or semi-rigid material is selected from the group consisting of polydimethylsiloxane (PDMS), PMMA (poly(methyl methacrylate)), PS (polystyrene), and PC (polycarbonate).

3. The module of claim 2, wherein the thin sheet of flexible or semi-rigid material has a thickness of approximately 8 μm.

4. The module of claim 2, wherein the thin sheet of flexible or semi-rigid material is molded to have channels and single-cell trapping structures on a front surface thereof.

5. A high-throughput yeast-aging analysis (HYAA) chip comprising a plurality of modules according to claim 1, wherein:
each module has a single medium inlet disposed on one side thereof, and a single medium outlet disposed on an opposite side thereof.

6. The HYAA chip of claim 5, wherein:
the modules are disposed one above the other on a surface of the thin sheet of flexible or semi-rigid material so that their inlets are all oriented to the one side of the sheet and their outlets are oriented to an opposite side of the sheet.

7. The HYAA chip of claim 5, wherein:
each module includes a plurality of channels branched from its single medium inlet and merged into its single medium outlet.

8. The HYAA chip of claim 7, wherein:
the channels are parallel with one another.

9. The HYAA chip of claim 5, further comprising:
a plurality of single-cell trapping structures disposed in each of the chambers, so as to be capture single cells from the flow of medium through the chamber.

10. The HYAA chip of claim 9, wherein:
the trapping structures are arranged in an array having a number of columns of trapping structures disposed vertically, one above the other, with spaces therebetween.

11. The HYAA chip of claim 10, wherein:
the trapping structures of one column are offset vertically from the trapping structures of an adjacent column to facilitate flow of medium and cells through the array.

12. The module of claim 1, wherein:
the trapping structures are cup-shaped, having an inlet and an outlet;
wherein the inlets of the trapping structures are larger than the outlets of the trapping structures.

13. The module of claim 12, wherein:
a width of the inlets of the trapping structures is approximately 6 μm;
a width of the outlets of the trapping structures is approximately 3 μm.

14. The module of claim 13, wherein:
a height of the trapping structures is approximately 5 μm.

15. The module of claim 14, wherein:
a plurality of trapping structures are arranged in an array of columns and rows; and
a column spacing is equal to or smaller than a row spacing to ensure high trapping efficiency and minimal channel obstruction by daughter cells removed from the trapped mother cells.

16. The module of claim 1, wherein:
the dimensions of the trapping structures are optimized to ensure that at least one of the following conditions are met: (i) only a single cell is captured in each trapping structure; (ii) the trapped cells are stably retained in the trapping structure during the entire course of an aging experiment; and (iii) the trapping structure does not pose a spatial constraint to cell size increase during aging.

17. A method of isolating and culturing a plurality of single cells, comprising:
providing a module comprising:
a medium inlet, a medium outlet, and a channel extending in fluid communication between the medium inlet and the medium outlet;
a chamber disposed the channel;
a plurality of single-cell trapping structures disposed in the chamber; and
a sample inlet for introducing cells into a flow of medium through the chamber; and
further comprising:
introducing a liquid medium continuously through the medium inlet;
injecting suspended yeast cells through the sample inlet; and
trapping individual mother cells in the single-cell trapping structures.

18. The method of claim 17, further comprising:
cultivating the trapped mother cells with continuous medium flow.

19. The method of claim 18, further comprising:
as the trapped cells mother cells grow and bud, and daughter cells are produced and detached from their mother cells, removing the daughter cells by the medium flow.

20. The method of claim 19, further comprising:
tracking the development of the mother cells over their entire lifespan in a single experiment using high-resolution multi-position time-lapse microscopy.
